(19) United States
(12) Patent Application Publication
(10) Pub. No.: US 2020/0115722 A1
(43) Pub. Date: Apr. 16, 2020

(54) BIOSYNTHESIS OF 1,3-BUTADIENE

(71) Applicant: Reliance Industries Limited, Mumbai, Maharashira (IN)

(72) Inventors: Sivaraman Balasubramaniam, Tamil Nadu (IN); Sneh Sanjay Badle, Maharashtra (IN); Swati Badgujar, Maharashtra (IN); Vinod Puthan Veetil, Kerala (IN); Vidhya Rangaswamy, Maharashtra (IN)

(21) Appl. No.: 16/087,602

(22) PCT Filed: Mar. 20, 2017
(86) PCT No.: PCT/US2017/051593

§ 371 Date: Sep. 21, 2018

(30) Foreign Application Priority Data
Mar. 21, 2016 (IN) 201621009837

(51) Int. Cl. C12P 5/02

(52) U.S. Cl. CPC C12P 5/026; C12Y 604/01002; C12Y 102/0101; C12Y 203/0118

(57) ABSTRACT

The present disclosure envisages a method for producing 1,3-butadiene by a biochemical approach. The starting material used for the biosynthesis of 1,3-butadiene, i.e., malonyl-CoA, can be obtained by converting syngas to acetyl-CoA and further carboxylation to malonyl-CoA. The next step involves condensing malonyl-CoA and acetaldehyde via a decarboxylative Claisen condensation reaction, to obtain 3-hydroxybutyryl-CoA. Syngas, a byproduct of many industrial processes, is used here to produce 1,3-butadiene, which makes the method of the present disclosure economical, and produces a product having value addition.

### FIELD OF THE INVENTION

The present disclosure relates to the biosynthesis of 1,3-butadiene.

### BACKGROUND

1,3-Butadiene is a conjugated diene with the formula C4H6. It is an important industrial chemical used as a monomer in the production of synthetic rubber, including styrene-butadiene-rubber (SBR), polybutadiene (PB), styrene-butadiene latex (SBL), acrylonitrile-butadiene-styrene resins (ABS), nitrile rubber, and adiponitrile. 1,3-Butadiene is a co-product obtained from the steam cracking process of petrochemical-based feedstocks and purified via extractive distillation.

Due to the increasing demand for 1,3-butadiene, a need is felt for a simple and economic method for the production of 1,3-butadiene.

### SUMMARY OF THE INVENTION

Some of the objects of the present disclosure, which at least one embodiment herein satisfies, are as follows:

An object of the present disclosure to ameliorate one or more problems of the prior art or to at least provide a useful alternative.

Another object of the present disclosure to provide a method for producing 1,3-butadiene.

Still another object of the present disclosure is to provide a method for producing 1,3-butadiene using biological catalysts.

Yet another object of the present disclosure is to provide a method for producing 1,3-butadiene using chemical reagents.

Yet another object of the present disclosure is to provide a method for producing 1,3-butadiene which is environmentally-friendly and sustainable.

The present disclosure envisages a method for producing 1,3-butadiene involving the key intermediate 3-hydroxybutyryl-CoA. The method comprises microbial based decarboxylative claisen condensation reaction of malonyl-CoA and acetaldehyde in the presence of at least one acyltransferase enzyme and acyl carrier protein, to obtain 3-hydroxybutyryl-CoA, microbial based conversion of the 3-hydroxybutyryl-CoA to 1,3-butanediol in the presence of a dehydrogenase enzyme and NADH, and further dehydrating the 1,3-butanediol in the presence of at least one chemical reagent to obtain 1,3-butadiene. In one embodiment, the acyltransferase enzyme is beta-ketoacyl-ACP synthase III. The acetaldehyde can be obtained by the reduction of acetyl-CoA using acetaldehyde dehydrogenase.

The intermediate, 3-hydroxybutyryl-CoA can be converted to 1,3-butanediol in the presence of a dehydrogenase enzyme and nicotinamide adenine dinucleotide (NADH), and further dehydrating 1,3-butanediol in the presence of chemical reagents to obtain 1,3-butadiene. Alternatively, the intermediate, 3-hydroxybutyryl-CoA can be hydrolysed in the presence of thiolester hydrolase enzyme to obtain 3-hydroxybutanoic acid. The 3-hydroxybutanoic acid, is reduced in the presence of carboxylate reductase to obtain 3-hydroxybutanal, which is further reduced in the presence of an aldehyde reductase to obtain 1,3-butanediol. 1,3-butanediol is dehydrated in the presence of a catalyst to obtain 1,3-butadiene.

Typically, malonyl-CoA can be obtained by converting syngas to acetyl-CoA, and further conversion of acetyl-CoA to malonyl-CoA. In one embodiment, the conversion of syngas to acetyl-CoA is carried out in the presence of a microorganism comprising a tetrahydrofolate metabolism pathway.

### BRIEF DESCRIPTION OF THE DRAWINGS

FIG. 1 represents the PCR amplification of fabH gene.

FIG. 2 represents the screening for fabH positive clones.

FIG. 3 represents the PCR amplification of acp gene.

FIG. 4 represents the screening for acp positive clones.

FIG. 5 represents the PCR amplification of adhE (Cbei_3832) gene.

FIG. 6 represents the screening for adhE (Cbei_3832) positive clones.

FIG. 7 represents the PCR amplification of adhE (Cbei-0223), adhE (Cbei-0528), adhE (Cbei-1722), adhE (Cbei-0869), adhE (Cbei-2243), adhE (Cbei-2421), adhE (Cbei-2421) and adhE (Cbei_4053) genes from C. beijerinckii.

FIG. 8 represents the screening for adhE (Cbei-0223), adhE (Cbei-0528), adhE (Cbei-1722), adhE (Cbei-0869), adhE (Cbei-2243), adhE (Cbei-2421), and adhE (Cbei-2421) positive clones.

FIG. 9 represents the SDS-PAGE image of the purified overexpressed ACP, AdhE and FabH proteins.

FIG. 10 depicts an HPLC spectrum of 3-hydroxybutyryl-CoA obtained from FabH enzyme assay and overlaid with standard.

FIG. 11 depicts an HPLC spectrum of 1,3-butanediol obtained from cascade reaction (FabH and AdhE) and overlaid with control (without FabH and AdhE).

FIG. 12 depicts an HPLC spectrum of 1,3-butanediol obtained from cascade reaction (FabH and AdhE) and overlaid with standard 1,3-butanediol.

FIG. 13 depicts a GC-MS spectrum of 1,3-butanediol (standard), spiked, from cascade reaction (FabH and AdhE) and control (without FabH and AdhE).

FIG. 14 depicts the fragmentation pattern of GC-MS spectrum of 1,3-butanediol from cascade reaction (FabH and AdhE).

FIG. 15 depicts a standard plot for 1,3-butanediol by GC.

FIG. 16 depicts a standard plot for 1,3-butanediol by HPLC.

FIG. 17 depicts the GC-MS spectrum of 1,3-butadiene obtained by reacting 1,3-butanediol with H₃PO₄ and Zeolite NaY.

FIG. 18 depicts the standard plot for propene by GC.

FIG. 19 depicts the 1H-NMR spectrum of 1,3-butadiene obtained by reacting 1,3-butanediol with H₃PO₄ and Zeolite NaY, recorded in CDCl₃.

### DETAILED DESCRIPTION

1,3-Butadiene is an important industrial chemical used as a monomer in the production of synthetic rubber having various applications across the industry. Moreover, the strong increase in demand for the elastomeric products, drawn by the expansion of fields such as the automotive industry, has consequently led to an ever-increasing demand for 1,3-butadiene, used as raw material for the production of a large range of synthetic rubbers. 1,3-butadiene is largely produced by methods like naphtha cracking, direct dehydrogenation of n-butene, and oxidative dehydrogenation of n-butene. Among them, the naphtha cracking process is energy intensive and requires high infrastructure costs. Further, the naphtha cracking process is problematic because of production of several side products, so that the investment and operation for a naphtha cracker cannot be optimally matched with the production and demand of 1,3-butadiene, and other basic fractions besides 1,3-butadiene are excessively produced. Further, the naphtha cracking process being dependent on the petrochemical feedstocks, along with the increasing environmental concerns and the limited resources for producing 1,3-butadiene using chemical processes, the present disclosure envisages an alternative, environmentally-friendly and sustainable processes for the production of 1,3-butadiene. Particularly, there is envisaged a method for producing 1,3-butadiene by biocatalysis. More particularly, the present disclosure discloses a method for synthesizing 1,3-butadiene from syngas by biocatalysis.

In an aspect of the present disclosure, there is provided a method for producing 1,3-butadiene. The method involves condensing malonyl-CoA and acetaldehyde to obtain 3-hydroxybutyryl-CoA, and converting 3-hydroxybutyryl-CoA to 1,3-butadiene in the presence of at least one of an enzyme and/or at least one chemical reagent.

The condensation reaction between malonyl-CoA and acetaldehyde is a microbial based decarboxylative claisen condensation reaction, which is catalysed by acyl transferase to form 3-hydroxylbutyryl-CoA. In one embodiment, the acyltransferase enzyme is beta-ketoacyl-ACP synthase III.

In another embodiment, the acetaldehyde is obtained by reducing acetyl-CoA using a dehydrogenase, such as, acetaldehyde dehydrogenase.

In one embodiment, 3-hydroxybutyryl-CoA is converted to 1,3-butanediol using a microbial based conversion in the presence of a dehydrogenase enzyme, such as, aldehyde dehydrogenase, and NADH.

1,3-Butanediol, is then dehydrated in the presence of at least one chemical reagent to obtain 1,3-butadiene.

Typically, the chemical reagent is at least one selected from orthophosphoric acid, aqueous hydrogen iodide, trifluoroacetic acid, sulphuric acid, zeolites, ionic liquids, and combinations thereof. In accordance with the embodiments of the present disclosure, the chemical reagent is at least one selected from the group consisting of orthophosphoric acid, zeolite NaY, faujasite, and a combination thereof.

Alternatively, in another embodiment, 3-hydroxybutyryl-CoA is sequentially converted to 1,3-butadiene, in a series of steps. Initially, 3-hydroxybutyryl-CoA is hydrolysed in the presence of a thiolester hydrolase enzyme to obtain 3-hydroxybutanoic acid. In one embodiment, the thiolester hydrolase enzyme is thioesterase.

3-hydroxybutanoic acid is reduced in the presence of carboxylate reductase to obtain 3-hydroxybutanal.

3-hydroxybutanal is further reduced in the presence of an aldehyde reductase to obtain 1,3-butanediol.

1,3-butanediol, as described previously is dehydrated in the presence of at least one catalyst to obtain 1,3-butadiene.

The starting material, malonyl-CoA is obtained by converting syngas to acetyl-CoA in the presence of at least one microorganism; and further converting the acetyl-CoA to malonyl-CoA using a carbonyl donor in the presence of a carboxylase enzyme. Malonyl-CoA along with acetaldehyde is condensed to obtain 3-hydroxybutyryl-CoA, followed by conversion of 3-hydroxybutyryl-CoA to 1,3-butanediol. 1,3-Butadiene is then obtained by dehydrating 1,3-butanediol either by using a biocatalyst like a dehydratase enzyme or by using a dehydrating chemical reagent.

Scheme-I represents the method for synthesizing 1,3-butadiene in an embodiment of the present disclosure.

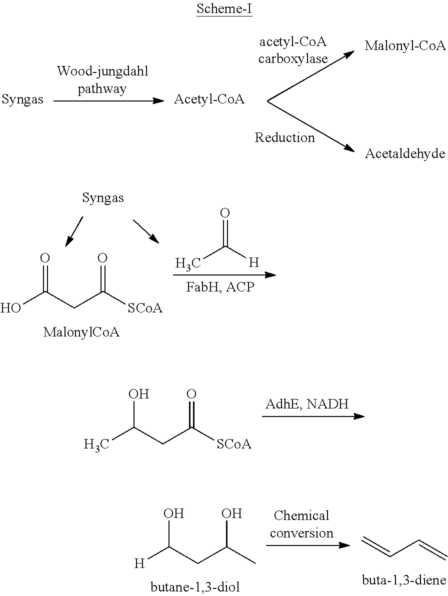

Synthesis gas (hereinafter referred to as syngas) is a mixture of hydrogen (H2) and carbon monoxide (CO). Syngas is a platform intermediate in the chemical and bio refining industries and has a vast number of uses. Syngas can be converted into alkanes, olefins, oxygenates, and alcohols. These chemicals can be blended into, or used directly as, diesel fuel, gasoline, and other liquid fuels. Carbon is fixed in anaerobic microorganisms from gaseous substrates like carbon monoxide or carbon dioxide via the Wood-Ljungdahl pathway. Carbon monoxide from syngas is also converted to acetyl-CoA through the Wood-Ljungdahl pathway utilizing tetrahydrofolate metabolism. Malonyl-CoA and acetaldehyde are derived from acetyl-CoA, which are the starting materials for producing 1,3-butadiene.

Typically, the microorganism comprises tetrahydrofolate metabolism pathway, and is selected from the group consisting of genera Acetitomaculum, Acetobacterium, Blautia, Clostridium, Eubacterium, Methanothermobacter, Moorella, Sporomusa, Syntrophococcus and Butyribacterium. The carbonyl donor can be selected from the group consisting of hydrogen carbonate and carboxylated biotin adducts. In one embodiment, the carboxylase enzyme is acetyl-CoA carboxylase.

Typically, live microorganisms which produce the enzyme(s) of interest or enzymes extracted from the microorganism can be used. In one embodiment, the live microorganisms used are genetically modified microorganisms.

The present disclosure envisages the use of syngas as a source for producing the starting materials used in the method for synthesizing 1,3-butadiene. Syngas, which is generally a byproduct of many industrial processes, can be used here to produce 1,3-butadiene, thus making the method of the present disclosure economical and produce a product having value addition.

The present disclosure is further described in light of the following laboratory scale experiments which are set forth for illustration purpose only and not to be construed for limiting the scope of the disclosure. These laboratory scale experiments can be scaled up to industrial/commercial scale and the results obtained can be extrapolated to industrial/commercial scale.

### Experimental Details

### Production of 1,3-butanediol and 1,3-butadiene in Accordance with the Present Disclosure Strains and Culture Conditions

Escherichia coli DH5α was used for cloning procedures, and was cultivated at 37° C. in Luria-Bertani (LB) medium containing 50 μg/mL kanamycin for selection with shaking at 220 rpm. The two clostridial strains, Clostridium acetobutylicum ATCC55383 and Clostridium beijerinckii ATCC 10132 were procured from American type culture collection and anaerobically grown in RCB medium (Reinforced clostridial broth). Genomic DNA from Escherichia coli K12 (Novagen, USA), C. acetobutylicum ATCC55383 and C. beijerinckii ATCC 10132 were isolated using Qiagen genomic DNA isolation kit. The FabH and Acp genes were amplified using E. coli genomic DNA. The genes Ca_adhE and Cbei_3832, Cbei_0223, Cbei_0528, Cbei_1722, Cbei_0869, Cbei_2243, Cbei_2421, and Cbei_4053 were amplified using C. acetobutylicum and C. beijerinckii genomic DNA respectively.

For shake-flask fermentation/overexpression, glycerol-stock was inoculated into a tube containing 10 mL of LB medium with respective antibiotic if required for overnight growth. The preculture was inoculated into a 500 mL of shake-flask containing 250 mL of fresh LB medium at an initial OD600 of 0.05, grown at 37° C. for 1-2 hours till OD of 0.5 is achieved and induced with 0.5 mM IPTG. Induced cultures were either incubated further at 37° C. for 4 hours or overnight at 20° C.

Chemical Reaction and Conditions

All reactions were carried out in oven dried glass wares or in cylindrical reaction vials with rubber cork. 1,3-Butanediol was obtained from Aldrich. All solvents were purchased from local suppliers and were of laboratory reagent grade. 1H-NMR (400 MHz) spectra was recorded in CDCl3 and chemical shifts are given in part per million (ppm). 1H-NMR spectra are referenced to CDCl3 (δ=7.26 ppm). The multiplicity is given as, s=singlet, d=doublet, m=multiplet and coupling constants J are reported in Hz.

### Analytical Methods

### Analysis of 3-hydroxybutyryl-CoA, 1,3-butanediol and 1,3-butadiene

3-Hydroxybutyryl-CoA and 1,3-butanediol were analyzed on HPLC. The conditions for the analysis of 3-hydroxybutyryl-CoA included using C18 column and isocratic mobile phase consisting of 100 mM ammonium acetate with 9% methanol and 0.1% formic acid at a flow rate of 0.8 mL min⁻¹. Column temperature was maintained at 25° C. The retention time for standard 3-hydroxybutyryl-CoA was 10.3 minutes.

The conditions for the analysis of 1,3-butanediol included the use of an anion exchange column and the mobile phase used was 5 mM sulfuric acid solution at a flow rate of 0.6 mL min⁻¹. Column temperature was maintained at 50° C. The retention time for standard 1,3-butanediol was 18.0 minutes.

For analysis of 1,3-butadiene, headspace sample of the sealed vial was withdrawn and injected on the GC system (DB-1 column, 100×0.5 mm, ID: 0.25 mm). Parameters for GC analysis were: FID detector temperature: 160° C., column temperature: 50° C., gas flow rate: 25 mL min-1. The retention time of standard 1,3-butadiene was 5.9 minutes.

Sample preparation for 1H-NMR: The head space aliquot was sampled and bubbled into the CDCl₃ solution in NMR tube which was kept at −10° C. The NMR tube was sealed and recorded instantly using Bruker NMR instrument.

### Construction of Recombinant Plasmids and Strains

All oligonucleotides were purchased from Eurofins. One Taq DNA polymerase, restriction enzymes and T4 DNA ligase were purchased from New England Biolabs (USA). DNA purification, plasmid isolation and PCR purification kits were purchased from Qiagen. Ni-NTA resin was purchased from Qiagen. All other reagents were of analytical grade. The pET-28a (+) plasmid and E. coli BL21 (DE3)/DH5α strains (Novagen, USA) were used as expression vector and host strains. The primers used in the present disclosure are summarized in Table-1.

TABLE 1
Primers used in the present disclosure

| | | | Size |
|---|---|---|---|
| S.No | Primer sequence 5′-3′ | Description | (bp) |
| SEQ ID | GGATCCATGAGCACTATCGAAGAACGCGTTA | FP_AE014075 K12_acp_ | 237 |
| NO: 1 | | BamHI | |
| SEQ ID | AAGCTTTTACGCCTGGTGGCCGTT | RP_AE014075 K12_acp_ | |
| NO: 2 | | HindIII | |
| SEQ ID | AAGTCTGGTACCATGTATACGAAGATTATTGGTAC | FP_AE014075 K12_fabH_ | 955 |
| NO: 3 | | KpnI | |
| SEQ ID | TGTCGACTCGAGATCCTAGAAACGAACCAGCG | RP_AE014075 Kl2_fabH_ | |
| NO: 4 | | XhoI | |
| SEQ ID | GAATTCATGAATAAAGACACACTAATACCTACAACTAAAGA | FP_adhE from Cbei_3832_ | 1407 |
| NO: 5 | | EcoRI | |
| SEQ ID | GTCGACTTAGCCGGCAAGTACACATCTTC | RP_adhE from Cbei_3832_ | |
| NO: 6 | | SalI | |
| SEQ ID | GAATTCATGAAAGTCACAACAGTAAAGGA | FP_adhE from Ca_adhE_ | 2632 |
| NO: 7 | | EcoRI | |
| SEQ ID | GTCGACTTAAGGTTGTTTTTTAAAACAATTTATATACATTCTT | RP_adhE from Ca_adhE_ | |
| NO: 8 | | SalI | |
| SEQ ID | GAATTCATGAAAGCATTAACAAAAACAAATCCAGGA | FP_adhE from Cbei_0223_ | 1032 |
| NO: 9 | | EcoRI | |
| SEQ ID | GTCGACTTAAGATCTTATTACTACTTTTAATTCTGTACCTT | RP_adhE from Cbei_0223_ | |
| NO: 10 | | SalI | |
| SEQ ID | GGATCCATGAAAGCAAGAGCAGCTGTTATAGCT | FP_adhE from Cbei_0528_ | 1248 |
| NO: 11 | | BamHI | |
| SEQ ID | GTCGACTTAAAGATTATTAAGTAAGAACTTCTCTGCCTCTT | RP_adhE from Cbei_0528_ | |
| NO: 12 | | SalI | |
| SEQ ID | GAATTCATGGCACGTTTTACTTTACCTAGGGAC | FP_adhE from Cbei_1722_ | 1167 |
| NO: 13 | | EcoRI | |
| SEQ ID | GTCGACCTATAGTTCAACCTTTGTCCCATAATATGTGCAT | RP_adhE from Cbei_1722_ | |
| NO: 14 | | SalI | |
| SEQ ID | GAATTCATGCCCTTAAAATACAAGAACCACATAGACTT | FP_adhE from Cbei_0869_ | 891 |
| NO: 15 | | EcoRI | |
| SEQ ID | GTCGACCTAACTATCAACCATAGTTCCACCATTTACATGAAG | RP_adhE from Cbei_0869_ | |
| NO: 16 | | SalI | |
| SEQ ID | GAATTCATGATTACAACAATTCAAAATGAGAAAGATATTTCACC | FP_adhE from Cbei_2243_ | 1038 |
| NO: 17 | | EcoRI | |
| SEQ ID | GTCGACTTACACAACTGTAAGCACGATTTTGCC | RP_adhE from Cbei_2243_ | |
| NO: 18 | | SalI | |
| SEQ ID | GAGCTCATGGAAAATTTTAATTATTCAATACCTACTAAAGTT | FP_adhE from Cbei_2421_ | 1164 |
| NO: 19 | | SacI | |
| SEQ ID | GTCGACCTAAAGTGCTGCTTTAAAAATCTTTAATATATCAT | RP_adhE from Cbei_2421_ | |
| NO: 20 | | SalI | |
| SEQ ID | GGATCCATGGTTTTTCAATTAGGTAGGAAAGGAGAGG | FP_adhE from Cbei_4053_ | 1152 |
| NO: 21 | | BamHI | |
| SEQ ID | GTCGACTTAGTACACTAATTGAATCAGTCTTTCCATACCAAAT | RP_adhE from Cbei_4053_ | |
| NO: 22 | | SalI | |

The 3-oxoacyl-[acyl-carrier-protein] synthase (fabH), and acyl carrier protein (acp) genes having a size of about 955 and 237 bp respectively were amplified from genomic DNA of E. coli K12. Similarly, different aldehyde-alcohol dehydrogenase (adhE) were amplified using C. acetobutylicum (Ca_adhE) and C. beijerinckii (Cbei_adhE) genomic DNA using One Taq polymerase. The genes were then cloned into pET30a (+) vector with respective restriction enzymes mentioned in above table. The recombinant plasmid carrying the fabH, and acp genes were validated by digesting with same restriction enzymes pairs i.e. BamHI/HindIII and KpnI/XhoI, respectively. The different adhE genes carrying pET30a (+) recombinant vector were also confirmed by restriction digestion using same pair of enzymes as mentioned in table 1.

Chemically competent E. coli DH5α and BL21 (DE) cells were used for transformation of plasmid DNA.

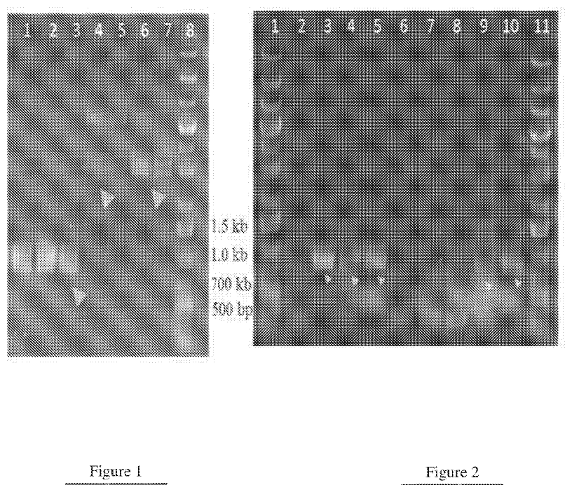

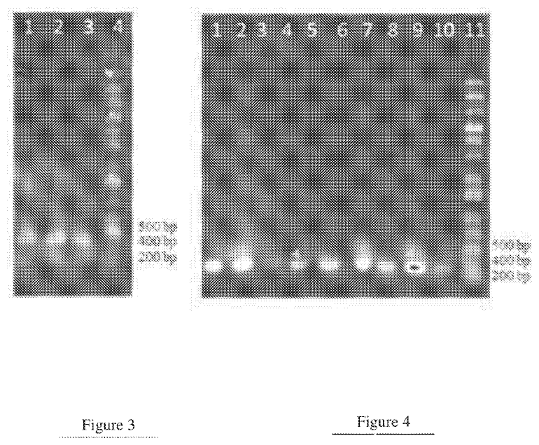

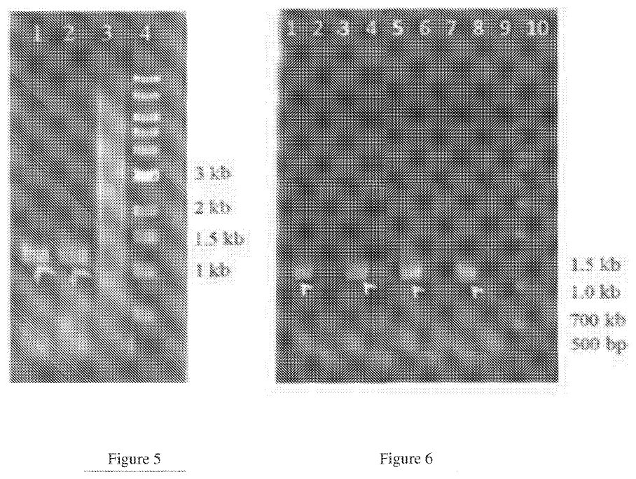

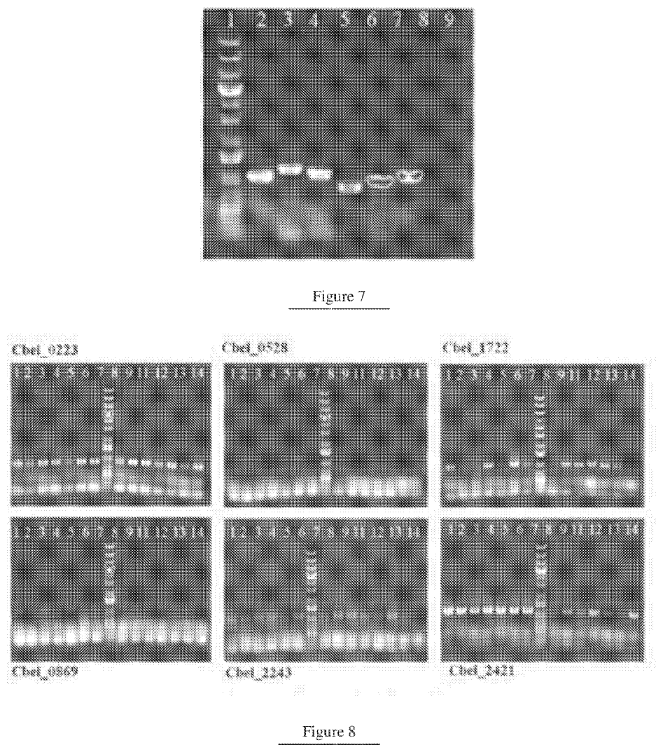

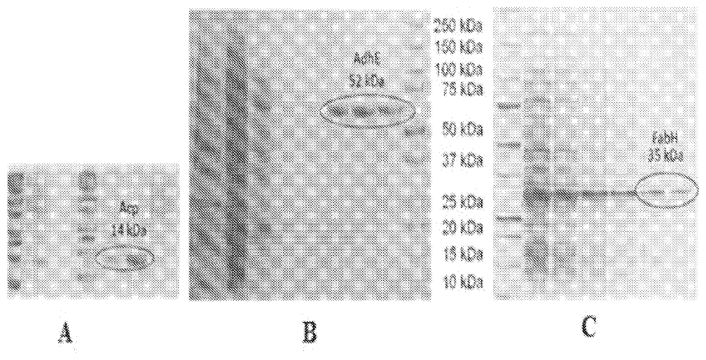

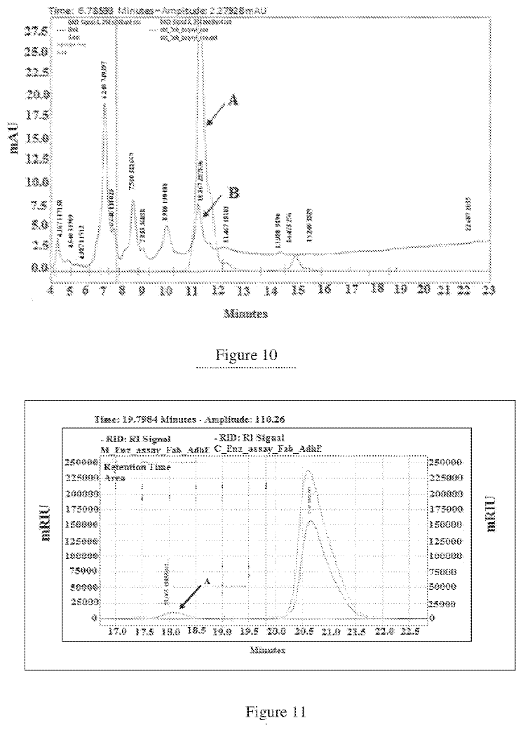

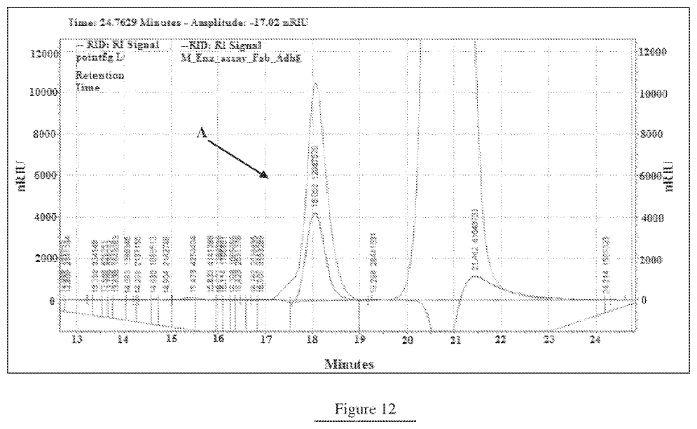

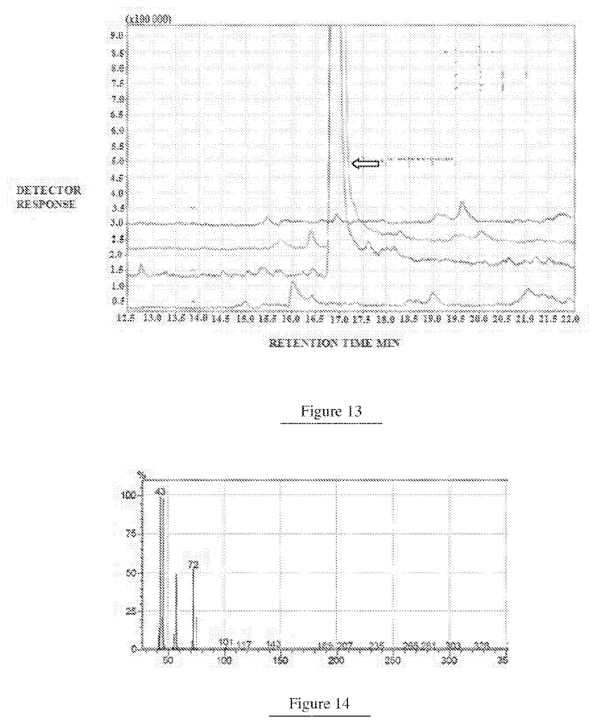

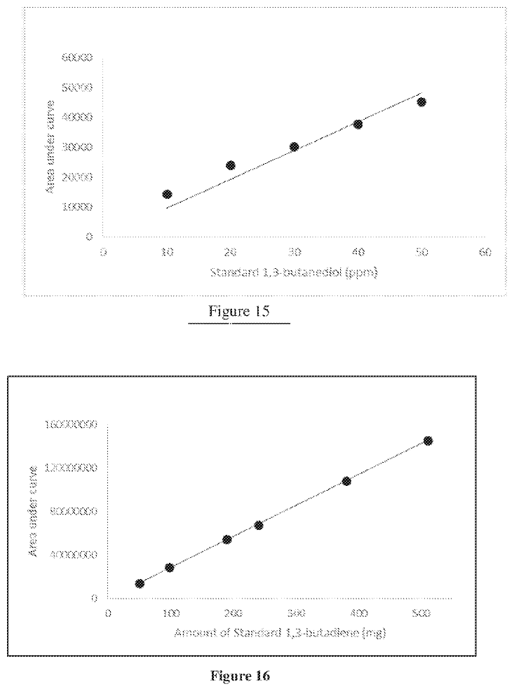

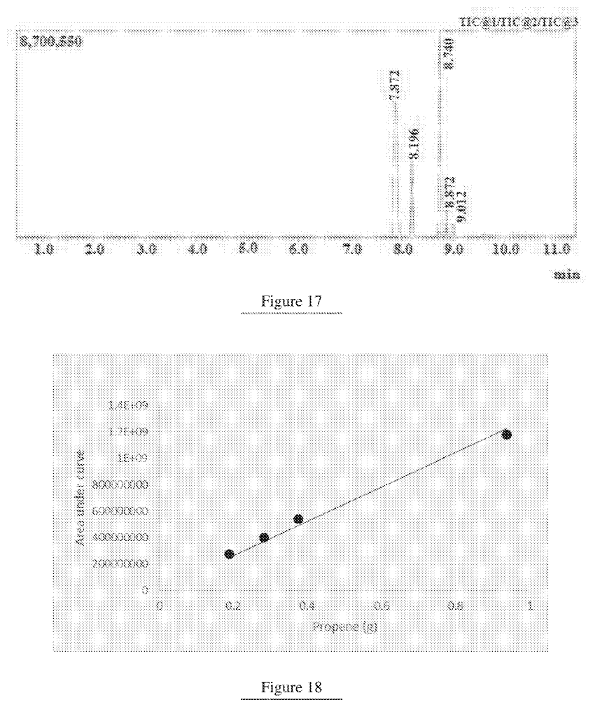

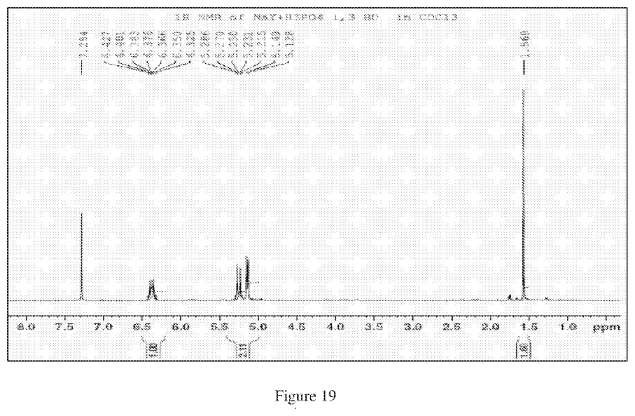

PCR amplifications of fabH, acp, and different adhE's (Cbei_adhE) from C. beijerinckii and (Ca_adhE) from C. acetobutylicum are shown in FIGS. 1, 3, 5, and 7, respectively. It is seen from FIGS. 1, 3, 5, and 7, that the PCR amplifications of the adhE from C. acetobutylicum (Ca_adhE) and C. beijerinckii (Cbei_4053) were not successful. After cloning into pET30 (+), the putative positives were analyzed by colony PCR as shown in FIGS. 2, 4, 6, and 8 for the respective genes. In FIG.-1, Lane 1 to 3 represents PCR amplified fabH product, Lane 4 and 5 represents pET30 plasmid, Lane 6 and 7 represents XhoI and KpnI digested pET30 (+) fragment and Lane-8 represents the gene ruler (1 kb). In FIG.-2, Lanes 2-10 represent colonies screened for fabH, and Lanes 1 and 11 represent the gene ruler (1 kb) ladder. In FIG.-3, Lanes 1 to 3 represent PCR amplified acp product, and Lane-4 represents the 100 bp ladder. In FIG.-4, Lane 1 to 10 represent colonies screened for acp, and Lane-11 represents the 100 bp ladder. In FIG.-5, Lane 1-2 represent PCR amplified adhE Cbei-3832 product, and Lane-4 represents 1 kb ladder. In FIG.-6, Lane 1-9 represent colonies screened for adhE (Cbei-3832), and Lane-10 represents 1 kb ladder. In FIG.-7, Lane-1 represents gene ruler (1 kb) Lane-2 represents PCR amplified adhE (Cbei-0223) product, Lane-3 represents PCR amplified adhE (Cbei-0528), Lane-4 represents PCR amplified adhE (Cbei-1722) product, Lane-5 represents PCR amplified adhE (Cbei-0869) product, Lane-6 represents PCR amplified adhE (Cbei-2243) product, Lane-7 represents PCR amplified adhE (Cbei-2421) product and Lane-8 represents PCR amplified adhE (Cbei-4053) product. FIG.-8, represents colonies screened for adhE (Cbei-0223), adhE (Cbei-0528), adhE (Cbei-1722), adhE (Cbei-0869), adhE (Cbei-2243), adhE (Cbei-2421), and adhE (Cbei-2421) on individual gel respectively. The arrows in FIGS. 1 to 8 point to the amplification bands observed at the correct size for the respective genes.

Expression and Purification of his-Tagged Protein

For expression tests, 10 mL of LB medium containing 50 μg mL−1 of kanamycin was inoculated with a freshly isolated bacterial colony of the host strain carrying a recombinant plasmid of pET30a-FabH, pET30a-ACP and pET30a-Cbei_3832 respectively. The inoculated cultures were incubated overnight at 37° C. and diluted 1:100 into 2 mL of fresh LB medium containing antibiotics with shaking until OD600 reached 0.6; induced with IPTG at a final concentration of 0.5 mM and were grown at 20° C. and 37° C. temperatures for 12 and 8 h respectively. 1 mL sample of induced cultures grown under different conditions were centrifuged and resuspended in 1 mL of lysis buffer. A portion was mixed with equal portion of 2×SDS loading buffer, and boiled for 10 min to prepare whole-cell lysate for expression analysis on SDS-PAGE. The samples for FabH and AdhE was run on 12% SDS-PAGE gel and ACP was run on 16% Tricin gel; gels were visualized by Coomassie Brilliant Blue R-250 staining. The target proteins were detected by comparison with protein standard markers. Optimum conditions were determined based on the observations from the gel.

For purification of the protein, growth and expression were carried out in 500 mL scale in optimal conditions identified by the expression tests. The cells harvested from 500 mL of culture grown and induced under optimum conditions were suspended in 10 mL of Lysis buffer (50 mM NaH₂PO₄, pH 8.0, 300 mM NaCl, and 5 mM imidazole) containing lysozyme (1 mg.mL−1) and cells were disrupted by sonication. Soluble and insoluble cell fractions were separated by centrifugation at 15000 rpm for 10 min in cold. Supernatants carrying the soluble fractions were mixed with Ni-NTA resin to purify fusion proteins according to manufacturer's manual. Bound His-tagged proteins were eluted in 1 mL of elution buffer containing 50 mmol NaH2PO4, 300 mmol NaCl, and 250 mmol imidazole, at pH 8.0. Equal volume of purified proteins were mixed with 2×SDS loading buffer and boiled for 10 min to prepare samples for SDS-PAGE.

The target proteins were detected by comparison with protein standard markers, the proteins were purified to >90% purity.

The SDS-PAGE of purified overexpressed proteins is shown in FIG.-9. The purified proteins bands of 14 kDa, 52 kDa and 35 kDa size corresponding to the enzymes ACP (A; 16% Tricin gel), AdhE (B; 12% SDS-PAGE), and FabH (C; 12% SDS-PAGE), respectively are shown in FIGS.-9A, 9B and 9C.

### Enzyme Assay:

### Activity of FabH

Enzyme activity of purified protein of FabH gene, β-ketoacyl-acyl carrier protein synthase III, was performed using the substrates malonyl-CoA and acetaldehyde. Acyl carrier protein was also purified and used in the enzyme reaction mixture. Cerulenin is a known inhibitor of FabH protein. The function of FabF is to facilitate chain elongation of fatty acids. As this was not desirable in the present disclosure, cerulenin was added. The total protein content as estimated by bicinchoninic acid assay (BCA) method for FabH was 200 mg.L−1. The reaction mixture constituted of malonyl-CoA 200 μM, acetaldehyde 250 μM, purified ACP protein 100 μg which was mixed thoroughly followed by the addition of cerulenin 250 μM (solution made in ethanol) and purified FabH 100 μg was added later. The enzyme mixture was incubated 37° C. for 2.5 hours. The mixture was then centrifuged at room temperature for 5 minutes at 8000 rpm. The supernatant was separated and tested for production of 3-hydroxybutyryl-CoA using HPLC and the result obtained is depicted in FIG.-10. In FIG.-10 peak A represents the overlay of the standard, and peak B represents the reaction peak of 3-hydroxybutyryl-CoA obtained from FabH enzyme.

Cascade Reaction with AdhE

The reaction mixture obtained from FabH enzyme assay was used as substrate for AdhE enzyme activity. The total protein content as estimated by bicinchoninic acid assay (BCA) method for AdhE was 15 mg.L−1.25 μL of 5 mM NADH solution and 100 μg of purified AdhE enzyme were added to 100 μL of FabH reaction mixture (containing 3-hydroxybutyryl-CoA). The reaction mixture was mixed thoroughly and incubated for 2.5 hours at 37° C. The enzymatic mixture was centrifuged for 5 min at 8000 rpm. The supernatant was analysed for the production of 1,3-butanediol using HPLC and GC-MS (FIG.-11 [Peak A represents 1,3-butanediol produced from the enzyme assay reaction at retention time of 18.0 minutes]; FIG.-12 [Peak A represents the overlay of reaction and standard 1,3-butanediol]); FIG.-13 and FIG.-14 [represents the GC-MS analysis and identification of 1,3-butanediol obtained from the reaction mixture]; Table-2 [quantification of 1,3-butanediol using GC-MS with FID]). In FIG.-13, 1 represents the control reaction (without FabH and AdhE), 2 represents the standard 1,3-butanediol (1 g/l), 3 represents the reaction spiked with 1,3-butanediol, and 4 represents the reaction mixture obtained from the cascade reaction: FabH followed by AdhE. FIG.-14 depicts a GC-MS spectrum of 1,3-butanediol obtained from cascade reaction. The data obtained from the analysis using HPLC and GC-MS proved the formation of 1,3-butanediol without any ambiguity.

### Control Experiments

Two sets of control experiments were carried out: 1) substrate control and 2) enzyme control.

Control experiments which did not contain either malonyl-CoA or acetaldehyde as a substrate were also performed. The HPLC analysis of these control reactions did not show the peak corresponding to 1,3-butanediol.

Control experiments which did not contain either FabH or ACP or AdhE as the enzyme were also performed but containing both malonyl-CoA or acetaldehyde as a substrate were carried out. The HPLC analysis of these control reactions did not show the peak corresponding to 1,3-butanediol.

TABLE 2
Area under curve (AUC) obtained for standard
1,3-butanediol analysed using GC

| Standard 1,3-butanediol (ppm) | AUC |
|---|---|
| 10 | 14400 |
| 20 | 24109 |
| 30 | 30225 |
| 40 | 37766 |
| 50 | 45307 |

100 μl of sample was diluted with 400 μl of methanol and the standard plot of 1,3-butanediol was prepared to determine the concentration of 1,3-butanediol from the cascade enzyme assay as depicted in FIG.-15. The concentration of 1,3-butanediol in the cascade enzyme assay was found to be 45 ppm based on AUC values.

### Chemical Conversion of 1,3-butanediol (1,3 BDO) to 1,3-butadiene (1,3 BD)

### General Procedure:

To 1 g of 1,3-butanediol, 85% H3PO4 or zeolite NaY or a combination of both H3PO4 and zeolite NaY was added and heated in a sealed reaction vial. The progress of the reaction was monitored using gas chromatography (GC) by injecting a small aliquot (0.1 ml) of samples drawn from the headspace of the reaction vessels. GC revealed presence of two peaks one at retention time of 5.5 minutes and other at retention time of 5.9 minutes. The peak corresponding to retention time of 5.9 minutes matched with that of the standard 1,3-butadiene. The headspace was analyzed using GC-MS and the product 1,3-butadiene was confirmed along with the presence of propene and trace amounts of E or Z-butenes based on the mass spectra (FIG. 17). The peak at retention time 5.5 minutes in GC spectrum corresponds to propene.

Optimization studies for the production of 1,3-butadiene was carried out by varying the reaction conditions and the reagents are given in Tables 3 to 10.

### 1. Using Orthophosphoric Acid as a Chemical Reagent

Optimization studies were carried out using 1 g of 1,3-butanediol by varying the reaction temperature, loadings of orthophosphoric acid and reaction time for the production of 1,3-butadiene.

TABLE 3
Effect of temperature

| | | | | GC observation | |
|---|---|---|---|---|---|
| | Loading of | | | Propene | 1,3-BD |
| Reaction | H₃PO₄ | Time | Temperature | (5.5 min) | (5.9 min) |
| 1 g 1,3 | 1 ml | 8 h | 90° C. | ~1% | 9% |
| BDO + | 1 ml | 8 h | 105° C. | 3% | 12% |
| 85% | 1 ml | 8 h | 125° C. | 6% | 12% |
| H₃PO₄ | 1 ml | 8 h | 150° C. | 5% | 8% |

TABLE 4
Effect of different loadings of H₃PO₄

| | | | | | GC observation | |
|---|---|---|---|---|---|---|
| | Loading of | | | | Propene | 1,3-BD |
| Reaction | H₃PO₄ | | Time | Temperature | (5.5 min) | (5.9 min) |
| 1 g 1,3 | 0.5 | ml | 8 h | 105° C. | ~1% | 9% |
| BDO + | 1 | ml | 8 h | 105° C. | 3% | 12% |
| 85% | 1.5 | ml | 8 h | 105° C. | 3% | 12% |
| H₃PO₄ | 2 | ml | 8 h | 105° C. | 5% | 15% |

TABLE 5
Effect of reaction time

| | | | | GC observation | |
|---|---|---|---|---|---|
| | Loading of | | | Propene | 1,3-BD |
| Reaction | H₃PO₄ | Time | Temperature | (5.5 min) | (5.9 min) |
| 1 g 1,3 | 1 ml | 8 h | 105° C. | ~2% | 12% |
| BDO + | 1 ml | 16 h | 105° C. | 3% | 22% |
| 85% | 1 ml | 24 h | 105° C. | 4% | 35% |
| H₃PO₄ | | | | | |

Optimization studies revealed that reacting 1,3-butanediol with 1 ml of orthophosphoric acid at 105° C. for 24 h gave best conversion resulting in 35% production of 1,3-butadiene. Reaction yields were derived from the standard graph based on the AUC values obtained from GC (FIG.-16 and FIG.-18).

### 2. Using Zeolite NaY as a Chemical Reagent

Optimization studies were carried out using 1 g of 1,3-butanediol by varying the reaction temperature, loadings of NaY and reaction time for the production of 1,3-butadiene.

TABLE 6
Effect of temperature

| | | | | GC observation | |
|---|---|---|---|---|---|
| | Loading of | | | Propene | 1,3-BD |
| Reaction | NaY | Time | Temperature | (5.5 min) | (5.9 min) |
| 1 g 1,3 | 500 mg | 8 h | 105° C. | negligible | 2% |
| BDO + | 500 mg | 8 h | 125° C. | ~1% | 9% |
| NaY | 500 mg | 8 h | 135° C. | ~1% | 11% |
| | 500 mg | 8 h | 150° C. | 5% | 12% |

TABLE 7
Effect of different loadings of NaY

| | | | | | GC observation | |
|---|---|---|---|---|---|---|
| | Loading of | | | | Propene | 1,3-BD |
| Reaction | NaY | | Time | Temperature | (5.5 min) | (5.9 min) |
| 1 g 1,3 | 100 | mg | 8 h | 135° C. | ~1% | 3% |
| BDO + | 250 | mg | 8 h | 135° C. | 3% | 8% |
| NaY | 500 | mg | 8 h | 135° C. | ~1% | 11% |
| | 1 | g | 8 h | 135° C. | 9% | 15% |

TABLE 8
Effect of reaction time

| | | | | GC observation | |
|---|---|---|---|---|---|
| | Loading of | | | Propene | 1,3-BD |
| Reaction | NaY | Time | Temperature | (5.5 min) | (5.9 min) |
| 1 g 1,3 | 500 mg | 8 h | 135° C. | ~1% | 11% |
| BDO + | 500 mg | 16 h | 135° C. | 4% | 15% |
| NaY | 500 mg | 24 h | 135° C. | 9% | 24% |

Optimization studies revealed that reacting 1,3-butanediol with 500 mg of zeolite NaY at 135° C. for 24 h gave best conversion resulting in 24% of 1,3-butadiene production. Reaction yields were derived from the standard graph based on the AUC values obtained from GC (FIG.-16 and FIG.-18).

### 3. Using Mixture of H₃PO4 and Zeolite NaY as Chemical Reagents

Loadings of orthophosphoric acid was varied from 0.25 mL to 1.0 mL for 1 g of 1,3-butanediol with 500 mg of NaY to optimize 1,3-butadiene production.

TABLE 9
Effect of different loadings of H₃PO₄ with 500 mg NaY

| | | | | GC observation | |
|---|---|---|---|---|---|
| | Loading of | | | Propene | 1,3-BD |
| Reaction | H₃PO₄ | Time | Temperature | (5.5 min) | (5.9 min) |
| 1 g 1,3 | 0.25 ml | 8 h | 135° C. | ~1% | 12% |
| BDO + | 0.50 ml | 8 h | 135° C. | ~4% | 16% |
| 500 mg | 0.75 ml | 8 h | 135° C. | ~5% | 14% |
| NaY | 1.0 ml | 8 h | 135° C. | ~7% | 12% |

Similarly, loadings of NaY was varied from 100 mg to 400 mg for 1 g of 1,3-butanediol with 0.5 mL of orthophosphoric acid to optimize 1,3-butadiene production.

TABLE 10
Effect of different loadings of NaY with 0.5 ml of H₃PO₄

| | | | | GC observation | |
|---|---|---|---|---|---|
| | Loading of | | | Propene | 1,3-BD |
| Reaction | NaY | Time | Temperature | (5.5 min) | (5.9 min) |
| 1 g 1,3 | 100 mg | 8 h | 135° C. | ~1% | 7% |
| BDO + | 200 mg | 8 h | 135° C. | ~1% | 15% |
| 0.5 ml | 300 mg | 8 h | 135° C. | 3% | 12% |
| H₃PO₄ | 400 mg | 8 h | 135° C. | 5% | 14% |

After optimizing the reaction temperature and loading, it was found that reacting 1 g of 1,3-butanediol with a mixture of 0.5 ml of orthophosphoric acid and 200 mg of zeolite NaY at 135° C. for 24 h gave best conversion resulting in 64% of 1,3-butadiene. Reaction yields were derived from the standard graph based on the AUC values obtained from GC (FIG.-16 and FIG.-18).

FIG.-17 depicts the mass spectrum of 1,3-butadiene obtained by reacting 1,3-butanediol with H3PO4 and zeolite NaY. It reveals the presence of two major peaks, one corresponds to propene (m/z=42) and other to 1,3-butadiene (m/z=54) along with trace amount of other impurities (Z or E 2-butene), for which the details are shown in table 11.

TABLE 11
Details of GC-MS spectra of 1,3-butadiene obtained by
reacting 1,3-butanediol with H₃PO₄ and zeolite NaY

| | Retention | Compound | Molecular |
|---|---|---|---|
| S. No | time (min) | name | weight |
| 1 | 7.8 | Nitrogen | 28 |
| 2 | 8.5 | Propene | 42 |
| 3 | 8.7 | 1,3-butadiene | 54 |
| 4 | 8.9 | Z or E-2-butene | 56 |
| 5 | 9.0 | Z or E -2-butene | 56 |

### Scale Up Reaction for the Conversion 1,3-butanediol to 1,3-butadiene

To 10 g of 1,3-butanediol, 5 ml of 85% H₃PO₄ and 2 g of zeolite NaY was added. The reaction vessel was sealed and heated at 135° C. for 24 h. The heating was stopped and the reaction mixture was brought to 65° C. The reaction mixture was analyzed using gas chromatography (GC) by injecting a small aliquot (0.1 mL) of samples drawn from the headspace of the reaction vessel. GC revealed presence of two peaks, one for propene (retention time=5.5 minutes) and other for 1,3-butadiene (retention time=5.9 minutes). The yield of 1,3-butadiene was 66% and that of propene was 4% as inferred from the standard graphs (FIG.-16 and FIG.-18). The residual 1,3-butanediol, which was analyzed using HPLC and was found to be 20%, which is 2 g. The product was characterized using 1H-NMR (400 MHz, CdCl₃): δ=6.23-6.33 (m, 2H), 5.13-5.19 (m, 2H), 5.03-5.08 (m, 2H) ppm, as depicted in FIG.-19.

### TECHNICAL ADVANCEMENTS

The present disclosure described herein above has several technical advantages including, but not limited to, the realization of a method for producing 1,3-butadiene via biocatalysis which is environmental friendly and economical.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

The numerical values given for various physical parameters, dimensions and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. A method for producing 1,3-butadiene, wherein said method comprises the following steps:
a) microbial based decarboxylative claisen condensation reaction of malonyl-CoA and acetaldehyde in the presence of at least one acyltransferase enzyme and acyl carrier protein, to obtain 3-hydroxybutyryl-CoA;
b) microbial based conversion of said 3-hydroxybutyryl-CoA to 1,3-butanediol in the presence of a dehydrogenase enzyme and NADH; and
c) dehydrating said 1,3-butanediol in the presence of at least one chemical reagent to obtain 1,3-butadiene.

2. The method as claimed in claim 1, wherein said acyltransferase enzyme is beta-ketoacyl-ACP synthase III.

3. The method as claimed in claim 1, wherein said acetaldehyde is obtained by reducing acetyl-CoA using acetaldehyde dehydrogenase.

4. The method as claimed in claim 1, wherein said chemical reagent is at least one selected from orthophosphoric acid, aqueous hydrogen iodide, trifluoroacetic acid, sulphuric acid, zeolites, ionic liquids, and combinations thereof.

5. The method as claimed in claim 4, wherein said chemical reagent is at least one selected from the group consisting of orthophosphoric acid, zeolite NaY or Faujasite, and a combination thereof.

6. The method as claimed in claim 1, wherein said malonyl-CoA is obtained by the following steps:
a. converting syngas to acetyl-CoA in the presence of at least ca microorganism; and
b. converting said acetyl-CoA to said malonyl-CoA using a carbonyl donor in the presence of a carboxylase enzyme.

7. The method as claimed in claim 6, wherein said microorganism comprises tetrahydrofolate metabolism pathway, and is selected from the group consisting of genera Acetitomaculum, Acetobacterium, Blautia, Clostridium, Eubacterium, Methanothennobacter, Moorella, Sporomusa, Syntrophococcus and Butyribacterium.

8. The method as claimed in claim 6, wherein said carbonyl donor is selected from the group consisting of hydrogen carbonate and carboxylated biotin adducts.

9. The method as claimed in claim 6, wherein said carboxylase enzyme is acetyl-CoA carboxylase.


SEQUENCE LISTING (22 sequences)

SEQ ID NO: 1 - DNA, 31 bp, Artificial Sequence
  FP_AE014075 K12_acp_BamHI
ggatccatgagcactatcgaagaacgcgtta

SEQ ID NO: 2 - DNA, 24 bp, Artificial Sequence
  RP_AE014075 K12_acp_HindIII
aagcttttacgcctggtggccgtt

SEQ ID NO: 3 - DNA, 35 bp, Artificial Sequence
  FP_AE014075 K12_fabH_ KpnI
aagtctggtaccatgtatacgaagattattggtac

SEQ ID NO: 4 - DNA, 32 bp, Artificial Sequence
  RP_AE014075 K12_fabH_ XhoI
tgtcgactcgagatcctagaaacgaaccagcg

SEQ ID NO: 5 - DNA, 41 bp, Artificial Sequence
  FP_adhE from Cbei_3832_EcoRI
gaattcatgaataaagacacactaatacctacaactaaaga

SEQ ID NO: 6 - DNA, 29 bp, Artificial Sequence
  RP_adhE from Cbei_3832_SalI
gtcgacttagccggcaagtacacatcttc

SEQ ID NO: 7 - DNA, 29 bp, Artificial Sequence
  FP_adhE from Ca_adhE_EcoRI
gaattcatgaaagtcacaacagtaaagga

SEQ ID NO: 8 - DNA, 44 bp, Artificial Sequence
  RP_adhE from Ca_adhE_SalI
gtcgacttaaggttgttttttaaaacaatttatatacatttctt

SEQ ID NO: 9 - DNA, 36 bp, Artificial Sequence
  FP_adhE from Cbei_0223 _EcoRI
gaattcatgaaagcattaacaaaaacaaatccagga

SEQ ID NO: 10 - DNA, 41 bp, Artificial Sequence
  RP_adhE  from Cbei_0223 _SalI
gtcgacttaagatcttattactacttttaattctgtacctt

SEQ ID NO: 11 - DNA, 33 bp, Artificial Sequence
  FP_adhE from Cbei_0528 _BamHI
ggatccatgaaagcaagagcagctgttatagct

SEQ ID NO: 12 - DNA, 41 bp, Artificial Sequence
  RP_adhE  from Cbei_0528 _SalI
gtcgacttaaagattattaagtaagaacttctctgcctctt

SEQ ID NO: 13 - DNA, 33 bp, Artificial Sequence
  FP_adhE from Cbei_1722 _EcoRI
gaattcatggcacgttttactttacctagggac

SEQ ID NO: 14 - DNA, 40 bp, Artificial Sequence
  RP_adhE from Cbei_1722 _SalI
gtcgacctatagttcaacctttgtcccataatatgtgcat

SEQ ID NO: 15 - DNA, 38 bp, Artificial Sequence
  FP_adhE from Cbei_0869 _EcoRI
gaattcatgcccttaaaatacaagaaccacatagactt

SEQ ID NO: 16 - DNA, 42 bp, Artificial Sequence
  RP_adhE from  Cbei_0869 _SalI
gtcgacctaactatcaaccatagttccaccatttacatgaag

SEQ ID NO: 17 - DNA, 44 bp, Artificial Sequence
  FP_adhE from Cbei_2243 _EcoRI
gaattcatgattacaacaattcaaaatgagaaagatatttcacc

SEQ ID NO: 18 - DNA, 33 bp, Artificial Sequence
  RP_adhE from Cbei_2243 _SalI
gtcgacttacacaactgtaagcacgattttgcc

SEQ ID NO: 19 - DNA, 42 bp, Artificial Sequence
  FP_adhE from Cbei_2421 _SacI
gagctcatggaaaattttaattattcaatacctactaaagtt

SEQ ID NO: 20 - DNA, 41 bp, Artificial Sequence
  RP_adhE from Cbei_2421 _SaII
gtcgacctaaagtgctgctttaaaaatctttaatatatcat

SEQ ID NO: 21 - DNA, 37 bp, Artificial Sequence
  FP_adhE from Cbei_4053 _BamHI
ggatccatggtttttcaattaggtaggaaaggagagg

SEQ ID NO: 22 - DNA, 43 bp, Artificial Sequence
  RP_adhE from Cbei_4053 _SalI
gtcgacttagtacactaattgaatcagtctttccataccaaat